(19) United States
(12) Patent Application Publication
(10) Pub. No.: US 2002/0041902 A1
(43) Pub. Date: Apr. 11, 2002

(54) Fluoride as a chemoprotective and chemotheraputic agent for cancer in mammals

(76) Inventor: Gregory Gene Steiner, Honolulu, HI (US)

Correspondence Address: Gregory Gene Steiner, P.O. Box 61515, Honolulu, HI, 96839, US

(21) Appl. No.: 09/952,466

(22) Filed: Sep. 14, 2001

Related U.S. Application Data
(60) Provisional application No. 60/236,951, filed on Sep. 29, 2000.

(51) Int. Cl. A61K 33/14; A61K 33/06; A61K 31/59

(52) U.S. Cl. 424/673; 424/682; 514/167

(57) ABSTRACT

Administered cancer chemopreventive and chemotherapeutic compositions of matter are disclosed. The compositions and method utilize fluoride as a cancer chemopreventive and chemotherapeutic agent in mammals, including humans. Additionally, novel compositions are disclosed comprising an effective amount of fluoride in combination with calcium or vitamin D or both designed to maintain skeletal health.

### FIELD OF THE INVENTION

The present invention relates to cancer chemopreventive and cancer chemotherapeutic compositions and methods. More particularly, the present invention relates to cancer chemoprevention and chemotherapeutic compositions utilizing fluoride as a chemopreventive and chemotherapeutic agent.

### BACKGROUND OF THE INVENTION

Cancer prevention is now a well-established medical science. Chemoprevention has been described as the intervention with specific agents to prevent, inhibit or reverse carcinogenesis before malignancy. At this time there is a concerted effort to find effective chemopreventive agents for cancer and also to subject these agents to mechanistic studies to determine their mode of action.

Chemotherapy is the treatment of cancerous lesions with the goal of slowing or stopping cancer cell division and spread. Chemotherapy for cancer traditionally used cytotoxic drugs with the intent of killing the cancer cells before the host succumbed to the cancer or the chemotherapy.

Fluoride is a common component of chemopreventive and chemotherapeutic agents. However, fluoride has not been recognized as having chemoprotective or chemotherapeutic properties. Fluoride has not been proposed as the active ingredient in chemopreventive or chemotherapeutic agents.

### SUMMARY OF THE INVENTION

In accordance with one aspect of the invention a composition effective in the prevention and treatment of cancer includes the novel use of fluoride or a pharmaceutically acceptable salt thereof in an amount effective to elicit a chemoprotective or chemotherapeutic response in mammals.

According to another aspect of the present invention, a method of eliciting a chemopreventive or chemotherapeutic effect in a mammal is the combination of fluoride with calcium in an effort to insure adequate calcium intake in order to prevent skeletal fluorosis and maintain skeletal health.

According to another aspect of the present invention, a method of eliciting a chemopreventive or chemotherapeutic effect in a mammal is the combination of fluoride with vitamin D in an effort to insure adequate vitamin intake in order to prevent skeletal fluorosis and maintain skeletal health.

According to another aspect of the present invention, a method of eliciting a chemopreventive and chemotherapeutic effect in a mammal is the combination of fluoride with calcium and vitamin D. Calcium and vitamin D are added to the chemopreventive and chemotherapeutic agent in an effort to insure adequate calcium and vitamin intake in order to prevent skeletal fluorosis and maintain skeletal health.

### DETAILED DESCRIPTION OF THE INVENTION

The correlation between latitude and cancer incidence rates is well established and has been recognized for many years. A number of explanations have been proposed for the effect of latitude on cancer incidence rates but no explanation has received widespread acceptance. Increased solar radiation in lower latitudes with the associated increased production of vitamin D has been correlated with a reduction in cancer incidence rates.¹ However, a recent publication found no correlation between cancer incidence rates and solar radiation.² 1. Garland C F, Garland F C, Gorman E D. Calcium and vitamin D. Their potential roles in colon and breast cancer prevention. Ann N Y Acad Sci 1999;889:107-19 2. Kampman E, Slattery M L, Caan B, Potter J D. Calcium, vitamin D, sunshine exposure, dairy products and colon cancer risk. Cancer, Causes and Control 2000; 11(5):459-66

In a study on the relationship of latitude and pancreatic cancer Kato et al reported a strong correlation between latitude and pancreatic cancer mortality and a strong inverse correlation with the average temperature.³ In Japan he found there was no difference between urban and rural pancreatic cancer mortality even thought there was a much higher fat intake in large cities. He concluded that there remains the possibility that factors related to latitude or average temperature other than diet may be involved in the occurrence of pancreatic cancer. 3. Kato I, Tajima K, Kuroishi T, Tominaga S. Latitude and pancreatic cancer. Jpn J Clin Oncol 1985;15(2):403-13

Two studies provide evidence that increased water consumption reduces bladder and colorectal cancer. Michaud et al studied the incidence of bladder cancer in 47,909 individuals over a 10-year period.⁴ They found that daily fluid intake was inversely correlated with the risk of bladder cancer. In conclusion they stated that a high fluid intake is associated with a decreased risk of bladder cancer in men. In a hospital based case-control study Tang et al found a strong inverse correlation between water intake and rectal cancer among men.⁵ The same trend was found for women but was not significant. 4. Michaud D S, Spiegelman D, Clinton S K, Rimm E B, Curhan G C, Willett W C, Giovannucci E L. Fluid intake and the risk if bladder cancer in men. N Engl J Med 1999;340(18): 1390-7 5. Tang R, Wang J, Lo S, Hseih L. Physical activity, water intake and risk of colorectal cancer in Taiwan: A hospital-based case control study. Int J Cancer 1999;82:484-489

Many carcinogens have been identified that produce an increased incidence of cancer.⁶ The discovery that fluoride reduces the incidence of caries was made in the early 1900's.⁷ By the 1950's municipal water departments were beginning to add fluoride to the water supply.⁷ As water was being fluoridated concerns that fluoride is carcinogenic were being voiced. This concern lead to studies to determine if fluoride in the drinking water is related to an increase in cancer incidence rates.⁹ 6. Begg C B. The search for cancer risk factors: when can we stop looking? Am J Public Health 2001;91(3):355-9 7. American Dental Association http://www.ada.org/public/topics/fluoride 9. Hoover R N, Mckay F W, Fraumeni J F. Fluoridated drinking water and the occurrence of cancer. J Natl Cancer 1976;57(4):757-68

In 1974 Nixon and Carpenter published a paper comparing standardized mortality ratios in relation to the amount of fluoride in the drinking water.⁸ They reported finding a statistically significant negative correlation between the standardized mortality rate and the fluoride content of the water. They concluded that all indications were that naturally occurring fluoride was likely to reduce cancer incidence. Hoover et al studied site-specific cancer mortality in Texas counties classified into four groups by fluoride concentration of the drinking water.⁹ Consistent trends were found for cancers of the buccal cavity, pharynx (males only) esophagus (males and females) and skin cancer (females). All trends indicated a reduction in cancer incidence with increased fluoride concentration. Multiple regression analysis revealed a statistically significant inverse correlation with the fluoride variable in four out of 64 tests of significance. Doll and Kinlen studied cancer incidence between 1950 and 1970 in cities with fluoridated water and in cities without fluoridation. When account was taken for age, sex, and ethnic group the ratio between observed cancer mortality and expected cancer mortality fell slightly in the cities with fluoridated water and did not change in the non-fluoridated cities.¹⁰ 8. Nixon J M, Carpenter R G. Mortality in areas containing natural fluoride in their water supplies, taking account of socioenvironmental factors and water hardness. Lancet 1974;2(7888):1068-71 9. Hoover R N, Mckay F W, Fraumeni J F. Fluoridated drinking water and the occurrence of cancer. J Natl Cancer 1976;57(4):757-68 10. Doll R, Kinlen L. Fluoridation of water and cancer mortality in the U.S.A. Lancet 1977;1(8025):1300-2

Mcguire et al in a case-control study of osteosarcoma patients found an inverse correlation between fluoride concentration and the incidence of osteosarcoma.¹¹ In 1995 Gelberg et al published a case-control study comparing fluoride exposure and childhood osteosarcoma.¹² They found a statistically significant correlation between increased fluoride intake and a decrease in the incidence of osteosarcoma for males. They proposed that fluoride might have a protective effect for males. 11. McGuire S M, Vanable E D, McGuire M H, Buckwalter J A, Douglass C W. Is there a link between fluoridated water and osteosarcoma? J Am Dental Assoc. 1991;122(4):39-45 12. Gelberg K H, Fitzgerald E F, Hwang S, Dubrow R. Fluoride exposure and childhood osteosarcoma: A Case-Control Study. American Journal of Public Health 1995;85(12):1678-83

In the 1970's a series of studies were carried out on normal cells treated with various agents known to initiate mutations by inducing chromosomal damage. Vogel reported a strong antimutagenic effect of fluoride on mutation induced by Trenimon and 1-phenyl-3,3-dimethyltriazene in Drosophila.¹³ In 1973 Obe and Slacik-Erben reported findings similar as Vogel and proposed that sodium fluoride exerts its antimutagenic action by suppressing events leading to chromosomal breakage.¹⁴ In 1976 Slacik-Erben et al reported that chromosomal aberrations induced by Trenimon revealed that pre, simultaneous and post-treatments of sodium fluoride significantly enhanced the frequency of undamaged mitosis.¹⁵ They interpreted their findings as an indication that sodium fluoride had significant antimutagenic activity. 13. Vogel E. Strong antimutigenic effects of fluoride on mutation induction by trenimon and 1-phenyl-3,3-dimethyltriazene in Drosophila Melanogaster. Mutation Research 1973;20:339-352 14. Obe G, Slacik-Erben R. Suppressive activity by fluoride on the induction of chromosome aberrations in human cells with alkylating agents in vitro. Mutation Research 1973;19:369-371 15. Slacik-Erben R, Obe G. The effects of sodium fluoride on DNA synthesis, mitotic indices and chromosomal aberrations in human leukocytes treated with Trenimon in vitro. Mutation Research 1976;37:253-266

Hirano et al studied the effects of fluoride on cultures of the osteosarcoma cell line UMR 106. The addition of 0.5 mM fluoride resulted in the induction of apoptosis and a decrease in cell proliferation.¹⁶ Anuradha et al reports that fluoride causes cell death in human leukemia (HL-60) cells by the activation of caspase-3 which in turn cleaves poly(ADP-ribose) polymerase leading to apoptosis.¹⁷ 16. Hirano S, Ando M. Fluoride mediates apoptosis in osteosarcoma UMR 106 and its cytotoxicity depends on the pH. Arch Toxicol 1997;72(1):52-8 17. Anuradha C D, Kanno S, Hirano S. Fluoride induces apoptosis by caspase-3 activation in human leukemia HL-60 cells. Arch Toxicol 2000;74(4-5):226-30.

Endemic fluorosis is a disease caused by high fluoride consumption resulting from high concentrations of fluoride in drinking water or food.¹⁸ Endemic fluorosis is identified by mottling of the teeth. Skeletal fluorosis can develop in areas with very high fluoride concentrations in the drinking water. In fluorosis, the fluoride ion replaces hydroxyl ions in bone apatite.¹⁹ 18. Singh A. Endemic fluorosis. Epidemiological, clinical and biochemical study of chronic fluorine intoxication in Punjab. Medicine 1961;42:229-246 19. Newman W F, Newman M W. The Chemical Dynamics of Bone Mineralization. University of Chicago Press, Chicago 1958

Endemic fluorosis and the associated elevated fluoride in drinking water forms a fluoride belt which stretches across the north and east of Africa, through the middle east, across Pakistan and India, into Southeast Asia and the south of China. In many areas on this belt endemic skeletal fluorosis has become a major health issue requiring defluoridation of the drinking water. When plotted on the world map there appears to be an association between endemic fluorosis and reduced cancer incidence rates. The presence of endemic fluorosis allows us to identify areas with very high levels of fluoride in the drinking water. Latitude, temperature and the fluoride concentration in the drinking water supply for areas with very high cancer incidence rates and areas with very low cancer incidence rates were compared in an effort to determine if cancer incidence rates are correlated with latitude, temperature and fluoride concentration.

Cancer incidence in five continents Vol. VII²⁰ was used as a reference to form the comparison groups very high cancer incidence rate and very low cancer incidence rate. Age-standardized rates for all sites but 173 was chosen because many cancer reporting stations do not fully report squamous cell and basal cell carcinoma of the skin.²¹ 20. Parkin D M, Whelan S L, Ferlay J, Raymond L, Young J, eds. Cancer incidence in five continents. Volume VII. IARC Sci Publ 1997;(143):I-xxxiv,1-1240 21. Weinstock M A, Bogaars H A, Ashly M, Litle V, Bilodeau E, Kimmel S. Inaccuracies in certification of nonmelanoma skin cancer deaths. Am J Public Health 1992;82(2):278-81

The very high cancer incidence rate group includes those cancer-reporting stations with a male cancer incidence rate over 300 per 100,000 and a female cancer incidence rate over 250 per 100,000 (Table 1). The cancer incidence rate for males and females was added together to provided a total cancer incidence rate for each reporting station. The very low cancer incidence rate group includes cancer-reporting stations with male and female cancer incidence rates under 160 per 100,000 (Table 2). Again the cancer incidence rate for males and females was added together to provided a total cancer incidence rate.

TABLE 1
VERY HIGH CANCER INCIDENCE RATE GROUP
Age-standardized cancer incidence rate per 100,000 population²⁰

| Location | Male | Fem. | Total |
|---|---|---|---|
| San Francisco, Black | 465 | 286 | 751 |
| Detroit, Black | 464 | 278 | 742 |
| San Francisco, White | 408 | 305 | 713 |
| Atlanta, Black | 450 | 253 | 703 |
| New Zealand, Maori | 360 | 340 | 700 |
| Connecticut, Black | 425 | 273 | 698 |
| Detroit, White | 401 | 294 | 695 |
| Hawaii, White | 398 | 295 | 693 |
| New Orleans, Black | 418 | 271 | 689 |
| Los Angeles, Black | 425 | 262 | 687 |
| Seattle | 395 | 290 | 685 |
| Los Angeles, White | 373 | 304 | 677 |
| Italy, Trieste | 414 | 256 | 670 |
| New Orleans, White | 389 | 273 | 662 |
| Atlanta, White | 384 | 273 | 657 |
| Uruguay, Montevideo | 317 | 255 | 626 |
| Central Calif., White | 351 | 271 | 622 |
| Canada, Yukon | 326 | 295 | 621 |
| Iowa | 348 | 271 | 619 |
| New Mexico, White | 354 | 261 | 615 |
| Canada, Nova Scotia | 338 | 268 | 606 |
| Canada, Ontario | 326 | 261 | 587 |
| Canada, Manatoba | 325 | 261 | 586 |
| Canada, Prince Edward Is | 322 | 260 | 582 |
| Australia, South | 324 | 251 | 575 |
| Scotland, West | 317 | 256 | 573 |
| Austria, Tyrol | 313 | 252 | 565 |
| Australia, West | 312 | 252 | 564 |
| Scotland | 306 | 257 | 563 |
| Canada, British Columbia | 307 | 254 | 561 |

TABLE 2
VERY LOW CANCER INCIDENCE RATE GROUP
Age-standardized cancer incidence rate per 100,000 population²⁰

| Location | Male | Fem. | Total |
|---|---|---|---|
| The Gambia* | 56 | 39 | 95 |
| India, Barshi etc. | 50 | 54 | 104 |
| Senegal, Dakar** | 76 | 75 | 151 |
| Algeria, Setif | 107 | 67 | 174 |
| India, Karunagappally | 108 | 80 | 194 |
| India, Trivandrum | 108 | 86 | 194 |
| MaJi, Bamako | 125 | 90 | 125 |
| India, Bangalore | 98 | 118 | 216 |
| Israel, Non-Jews | 128 | 94 | 222 |
| Singapore, Indian | 106 | 123 | 229 |
| Kuwait, Kuwaitis | 104 | 126 | 230 |
| Viet Nam, Hanoi | 143 | 89 | 232 |
| India, Madras | 117 | 130 | 247 |
| India, Bombay | 131 | 125 | 256 |
| Singapore, Malay | 145 | 133 | 278 |
| Lima, Peru | 124 | 151 | 275 |
| Thailand, Chiang Mai | 144 | 153 | 297 |
| New Mexico, Am Indian | 151 | 148 | 299 |
| Uganda, Kyadondo | 155 | 146 | 301 |

*Data from an earlier edition of Cancer incidence in five continents Vol VI 1992
**Data from an earlier edition of Cancer incidence in five continents Vol IV 1982

While there is no organized database for the world's drinking water supply most nations and many scientific investigations have published data on the world's drinking water. The fluoride concentration at the same site can very considerably according to whether the water source is surface water, shallow bore wells or deep bore wells. The concentration of fluoride can also very significantly from village to village. For this reason obtaining an exact figure for fluoride concentration in the drinking water for a cancer incidence-reporting station is difficult. Fluoride content in the water is related to geologic formations. Areas located in the same geologic formations will have similar fluoride levels in the ground water supply. For this reason, if a reporting station did not have a published fluoride level the fluoride concentration from an adjoining area was used for comparison.

Cancer incidence, latitude, temperature and fluoride concentration is listed for the very high and very low cancer incidence rate groups (tables 3 and 4). For those cancer-reporting stations with fluoridation the fluoride concentration after the addition of fluoride was used for calculating the correlation coefficient. The average maximum average temperature for the cancer incidence reporting stations was selected because daytime temperature should have the most significant effect on human physiology assuming people are insulated from the effects of nighttime temperatures.

TABLE 3
VERY HIGH CANCER INCIDENCE RATE WITH LATITUDE,
TEMPERATURE AND FLUORIDE IN THE DRINKING WATER

| Location | Total CI²⁰ | Lat.²⁰ | Temp²² | Nat. Fl* | Fluorosis** | Fluoridation |
|---|---|---|---|---|---|---|
| San Francisco, Black | 751 | 38 | 63 | <.4 mg/l | — | +²³ |
| Detroit, Black | 742 | 42 | 58 | <.4 mg/l | — | +²³ |
| San Francisco, White | 713 | 38 | 63 | <.4 mg/l | — | +²³ |
| Atlanta, Black | 703 | 34 | 72 | <.4 mg/l | — | +²³ |
| Maori | 700 | 40 | 59 | <.4 mg/l | — | +²³ |
| Connecticut, Black | 698 | 41 | 60 | <.4 mg/l | — | +²³ |
| Detroit, White | 695 | 42 | 58 | <.4 mg/l | — | +²³ |
| Hawaii, White | 693 | 22 | 84 | <.4 mg/l | — | —²⁴ |
| New Orleans, Black | 689 | 30 | 78 | <.4 mg/l | — | +²³ |
| Los Angeles, Black | 687 | 34 | 73 | <.4 mg/l | — | —²⁵ |
| Seattle | 685 | 47 | 59 | <.4 mg/l | — | +²³ |
| Los Angeles, White | 677 | 34 | 73 | <.4 mg/l | — | —²⁵ |
| Italy, Trieste | 670 | 45 | 63 | <.4 mg/l | — | —²⁶ |
| New Orleans, White | 662 | 30 | 78 | <.4 mg/l | — | +²³ |
| Atlanta, White | 657 | 34 | 72 | <.4 mg/l | — | +²³ |
| Uruguay, Montevideo | 626 | 35 | 70 | <.4 mg/l | — | +²³ |
| Central Calif., White | 622 | 36 | 76 | <.4 mg/l | — | +²³ |
| Canada, Yukon | 621 | 63 | 40 | <.4 mg/l | — | +²³ |
| Iowa | 619 | 42 | 60 | <.4 mg/l | — | +²³ |
| New Mexico, White | 615 | 35 | 70 | <.4 mg/l | — | +²³ |
| Canada, Nova Scotia | 606 | 49 | 48 | <.4 mg/l | — | +²³ |
| Canada, Ontario | 587 | 45 | 54 | <.4 mg/l | +²⁷ | +²³ |
| Canada, Manatoba | 586 | 49 | 47 | <.4 mg/l | — | +²³ |
| Canada, Prince Edward Is | 582 | 47 | 49 | <.4 mg/l | — | +²³ |
| Australia, South | 575 | 35 | 70 | <.4 mg/l | — | +²³ |
| Scotland, West | 573 | 57 | 52 | <.4 mg/l | — | —²⁸ |
| Austria, Tyrol | 565 | 46 | 57 | <.4 mg/l | — | —²⁹ |
| Australia, West | 564 | 32 | 75 | <.4 mg/l | — | +²³ |
| Scotland | 563 | 56 | 53 | <.4 mg/l | — | —²⁸ |
| Canada, British Columbia | 561 | 49 | 56 | <.4 mg/l | — | +²³ |

TABLE 4
VERY LOW CANCER INCIDENCE RATE WITH LATITUDE,
TEMPERATURE AND FLUORIDE CONCENTRATION
IN THE DRINKING WATER

| Location | Total CI²⁰ | Lat.²⁰ | Temp²² | Nat. Fl | Fluorosis | Fluoridation |
|---|---|---|---|---|---|---|
| The Gambia | 98* | 13 | 80 | 10 mg/l³⁰ | +³⁰ | —²³ |
| Barshi, Paranda, Bhum | 104 | 18 | 91 | 10 mg/l³¹ | +³¹ | —²³ |
| Senegal, Dakar | 151** | 13 | 80 | 10 mg/l³⁰ | +³⁰ | —²³ |
| Algeria, Setif | 174 | 35 | 72 | 4.0 mg/l³² | +³² | —²³ |
| India, Karunagappally | 188 | 9 | 86 | 5.1 mg/l^{33,34} | +³⁴ | —²³ |
| India, Trivandrum | 194 | 8 | 87 | 5.1 mg/l^{33,34} | +³⁴ | —²³ |
| Mali, Bamako | 215 | 13 | 92 | 10 mg/l³⁰ | +³⁰ | —²³ |
| India, Bangalore | 216 | 13 | 83 | 9.1 mg/l³⁴ | +³⁴ | —²³ |
| Israel, Non-Jews | 222 | 32 | 69 | 5.0 mg/l³⁵ | +³⁵ | —²³ |
| Singapore, Indian | 229 | 1 | 87 | 0.7 mg/l³⁶ | +³⁶ | +³⁶ |
| Kuwait, Kuwaitis | 230 | 29 | 90 | 0.4 mg/l³⁸ | +³⁸ | —²³ |
| Viet Nam, Hanoi | 232 | 21 | 80 | unknown | unknown | unknown |
| India, Madras | 247 | 13 | 90 | 3.3 mg/l³⁹ | +³⁹ | —²³ |
| India, Bombay | 256 | 19 | 88 | .32 mg/l³⁹ | +³⁹ | —²³ |
| Singapore, Malay | 278 | 1 | 87 | 0.7 mg/l³⁶ | +³⁶ | +³⁶ |
| Lima, Peru | 275 | 12 | 73 | unknown | unknown | unknown |
| Thailand, Chiang Mai | 297 | 16 | 89 | 5.0 mg/l⁴⁰ | +⁴¹ | —²³ |
| New Mexico, Am Indian | 299 | 35 | 70 | 4.1 mg/l⁴² | +⁴² | —²³ |
| Uganda, Kyadondo | 301 | 0 | 79 | 1.5 mg/l⁴³ | +⁴³ | —²³ |

*Data from an earlier edition of Cancer incidence in five continents Vol VI 1992
**Data from an earlier edition of Cancer incidence in five continents Vol IV 1982

The average latitude for the very high cancer incidence rate group is 41 degrees. The average latitude for the very low cancer incidence rate group is 16 degrees. The correlation between the cancer incidence rate and latitude is r=0.71.

The average maximum average temperature of the very high cancer incidence rate group is 63 degrees F. The average maximum average temperature of the very low cancer incidence rate group is 83 degrees F. The correlation between the cancer incidence rate and temperature is r=−0.87.

The average fluoride concentration of the very high cancer incidence rate group is 0.71 mg/l. The average fluoride concentration of the very low cancer incidence rate group is 5.0 mg/l. The correlation between the cancer incidence rate and the fluoride concentration in the drinking water is r=−0.75.

The very high cancer incidence rate group seldom reported endemic fluorosis. Endemic fluorosis was reported in 17 of 19 very low cancer incidence reporting areas. Information could not be found on the fluoride concentration or the incidence of fluorosis in Hanoi, Viet Nam or Lima, Peru.

Upon review of the available data, fluoride was found in high concentrations in virtually all very low cancer incidence rate areas and was found in low concentrations in all very high cancer incidence rate areas (tables 3 and 4).

Research has established a correlation between cancer incidence rates and the environmental factors latitude, temperature and water consumption. As a general rule cancer incidence rates increase with increasing latitude.²⁰ In this study an even stronger correlation is found between ambient temperature and the rate of cancer incidence. With the exception of Hawaii (the majority of drinking water in Hawaii lacks any detectable fluoride) none of the areas with high cancer incidence are found in the tropics (table 1). Almost all of the areas with low cancer incidence are found in the tropics (table 2). Humans are warm blooded and live with a very closely regulated internal temperature. A person in Senegal is the same temperature as a person in the Yukon. With this understanding latitude and temperature may only be indirectly correlated with the cancer incidence rate. 20. Parkin D M, Whelan S L, Ferlay J, Raymond L, Young J, eds. Cancer incidence in five continents. Volume VII. IARC Sci Publ 1997;(143):I-xxxiv,1-1240

The laborer in Senegal must perspire much more than a laborer in the Yukon to maintain the same body temperature. This fact suggests water consumption may be the reason latitude and temperature are related to cancer incidence rates. Because of a higher ambient temperature the person in lower latitudes will consume more water to maintain a stable body temperature. If a lower cancer incidence rate is correlated with increased water consumption, it is probable that the lower cancer incidence rate is more likely the result of something in the water.

In this study, fluoride concentration in the drinking water is inversely correlated with the cancer incidence rate. If fluoride were a chemoprotective agent it could explain why the cancer incidence rate is correlated with latitude, temperature and water consumption. The warmer the climate the more water is necessary to properly maintain body temperature through sweating. People living in lower latitudes would consume more water and therefore more fluoride. The finding that higher concentrations of fluoride are commonly found in the water in lower latitudes with warmer climates would accentuate the amount of fluoride consumed in lower latitudes.

New Mexico poses a unique comparison of drinking water fluoride concentration and the cancer incidence rate. The State of New Mexico has the distinction of being listed in both the very high cancer incidence rate group for non-Hispanic whites and also in the very low cancer incidence rate group for American Indians. With the exception of the major population centers of Albuquerque and Santa Fe many areas in the state report the fluoride concentration in the drinking water to be over 1.5 mg/l (mg/l=ppm).⁴⁴ Many water samples report fluoride concentrations above 5 mg/l and concentrations up to 20 mg/l have been documented.⁴⁵ The major population centers are located in areas of low fluoride with Albuquerque being fluoridated since 1974.⁴⁵ The population demographics historically were made up of the white population concentrated in the cities with low fluoride in the drinking water and the American Indian population located in the rural areas with high levels of fluoride in the drinking water. The non-Hispanic white population of New Mexico has a combined male and female cancer incidence rate of 615 per 100,000 people. The American Indian population of New Mexico has a combined male and female cancer incidence rate of 299 per 100,000 individuals. 44. US Geological Survey, Washington D.C., 1967 Fluoride concentration in the United States. 45. NMED Drinking Water Bureau, New Mexico Department of Health, www.nmenv.state.nm.us/gwb/haflor.htm

Children retain twenty percent of all fluoride consumed.⁴⁶ Because the body reabsorbs much of the fluoride released during bone remodeling the half-life of fluoride in the body has been estimated to be 20 years. If fluoride is a chemoprotective agent the long half-life of fluoride in the body could help explain why some cultures retain their low cancer incidence rates when they move to areas with high cancer incidence rates. A person living the first 10 years of his life in a high fluoride area such as India or Southern China will feasibly maintain an elevated plasma fluoride level for the remainder of his life. 46. Zohouri F V, Rugg-Gunn A J. Total fluoride intake and urinary excretion in 4-year-old Iranian children residing in low-fluoride areas. Br J Nutr 2000;83(1): 15-25

A factor affecting the fluoride intake of Asian populations is the fluoride found in tea.⁴⁷ Even in soils with low fluoride content the tea plant has the unique ability to concentrate fluoride in its leaves. Lakdawala et al analyzed the fluoride concentration of different foods and found tea to contain higher fluoride concentrations than any other food.⁴⁷ Tea leaves high in fluoride have been found to cause fluorosis when both the drinking water and the diet are low in fluoride.⁴⁸ 47. Lakdawala D R, Puenkar B D. Fluorine content of water and commonly consumed foods in Bombay and a study of the dietary fluorine intake. Dent Dialogue 1974;1(3):16-22 48. Cao J, Zhao Y, Lin J. Brick tea consumption as the cause of dental fluorosis among children from Mangol, Kazak and Yugu populations in China Food Chem Toxicol 1997;35(8):827-33

Bombay is located in a State with endemic fluorosis. However Bombay has very low cancer incidence and very low fluoride in the drinking water. With low fluoride in the drinking water and a very low cancer incidence rate Bombay tends to dispel fluoride as a chemoprotective agent. However, Lakdawala et al studied the fluoride content of the food and water consumed in Bombay and found high concentrations of fluoride in the food supply as a result of being grown in areas with high fluoride concentrations in the water.⁴⁷ 47. Lakdawala D R, Puenkar B D. Fluorine content of water and commonly consumed foods in Bombay and a study of the dietary fluorine intake. Dent Dialogue 1974;1(3):16-22

Many potential chemoprotective agents such a lutein, EGCG, kava and vitamin D have been studied.^{49,50,51,1} This study identifies fluoride as the agent responsible for reduced cancer incidence worldwide. 49. Vainio H, Rautalahti M. An international evaluation of the cancer preventive potential of carotenoids. Cancer Epidemiol Biomarkers Prev 1998;7(8):725-8 50. Fujiki H, Suganuma M, Okabe S, Sueoka E, Suga K, Inai K, Nakachi K, Kimura S. Mechanistic findings of green tea as cancer preventive for humans. Proc Soc Exp Biol Med 1999;220(4):225-8 51. Steiner G G. The correlation between cancer incidence and kava consumption. Hawaii Med J 2000;59(11):420-2 1. Garland C F, Garland F C, Gorman E D. Calcium and vitamin D. Their potential roles in colon and breast cancer prevention. Ann N Y Acad Sci 1999;889: 107-19

### MODE OF ACTION

Fluoride is known to significantly increase the bioactivity of molecules.⁵² Fluoride can activate enzymes by way of guanine nucleotide-binding proteins (G proteins), as in the activation of adenylate cyclase and polyphosphoinositide phosphodiesterase.⁵³ Fluoride is known to stimulate G proteins. 52. Welch J T, Eswarakrishman S. Fluorine in bioorganic chemistry. 1991 John Wiley and Sons Inc. New York 53. O'dell B L, Sunde R A, eds. Handbook of Nutritionally Essential Mineral Elements. 1997 Marcell Dekker Inc. New York

The addition of 0.5 mM fluoride to cultures of the ostosarcoma cell line UMR 106 resulted in the induction of apoptosis and a decrease in cell proliferation.¹⁶ Fluoride causes cell death in human leukemia (HL-60) cells by the activation of caspase-3 which in turn cleaves poly(ADP-ribose) polymerase leading to apoptosis.¹⁷ 16. Hirano S, Ando M. Fluoride mediates apoptosis in osteosarcoma UMR 106 and its cytotoxicity depends on the pH. Arch Toxicol 1997;72(1):52-8 17. Anuradha C D, Kanno S, Hirano S. Fluoride induces apoptosis by caspase-3 activation in human leukemia HL-60 cells. Arch Toxicol 2000;74(4-5):226-30.

It is the increase in bioactivity of molecules, the stimulation of G proteins and possibly other as yet unknown mechanisms whereby fluoride acts as a chemopreventive and chemotherapeutic agent.

### REFERENCES

1. Garland C F, Garland F C, Gorman E D. Calcium and vitamin D. Their potential roles in colon and breast cancer prevention. Ann N Y Acad Sci 1999;889:107-19

2. Kampman E, Slattery M L, Caan B, Potter J D. Calcium, vitamin D, sunshine exposure, dairy products and colon cancer risk. Cancer, Causes and Control 2000; 11(5):459-66

3. Kato I, Tajima K, Kuroishi T, Tominaga S. Latitude and pancreatic cancer. Jpn J Clin Oncol 1985;15(2):403-13

4. Michaud D S, Spiegelman D, Clinton S K, Rimm E B, Curhan G C, Willett W C, Giovannucci E L. Fluid intake and the risk if bladder cancer in men. N Engl J Med 1999;340(18): 1390-7

5. Tang R, Wang J, Lo S, Hseih L. Physical activity, water intake and risk of colorectal cancer in Taiwan: A hospital-based case control study. Int J Cancer 1999;82:484-489

6. Begg C B. The search for cancer risk factors: when can we stop looking? Am J Public Health 2001;91(3):355-9

7. American Dental Association http://www.ada.org/public/topics/fluoride

8. Nixon J M, Carpenter R G. Mortality in areas containing natural fluoride in their water supplies, taking account of socioenvironmental factors and water hardness. Lancet 1974;2(7888):1068-71

9. Hoover R N, Mckay F W, Fraumeni J F. Fluoridated drinking water and the occurrence of cancer. J Natl Cancer 1976;57(4):757-68

10. Doll R, Kinlen L. Fluoridation of water and cancer mortality in the U.S.A. Lancet 1977;1(8025):1300-2

11. McGuire S M, Vanable E D, McGuire M H, Buckwalter J A, Douglass C W. Is there a link between fluoridated water and osteosarcoma? J Am Dental Assoc. 1991;122(4):39-45

12. Gelberg K H, Fitzgerald E F, Hwang S, Dubrow R. Fluoride exposure and childhood osteosarcoma: A Case-Control Study. American Journal of Public Health 1995;85(12):1678-83

13. Vogel E. Strong antimutigenic effects of fluoride on mutation induction by trenimon and 1-phenyl-3,3-dimethyltriazene in Drosophila Melanogaster. Mutation Research 1973;20:339-352

14. Obe G, Slacik-Erben R. Suppressive activity by fluoride on the induction of chromosome aberrations in human cells with alkylating agents in vitro. Mutation Research 1973;19:369-371

15. Slacik-Erben R, Obe G. The effects of sodium fluoride on DNA synthesis, mitotic indices and chromosomal aberrations in human leukocytes treated with Trenimon in vitro. Mutation Research 1976;37:253-266

16. Hirano S, Ando M. Fluoride mediates apoptosis in osteosarcoma UMR 106 and its cytotoxicity depends on the pH. Arch Toxicol 1997;72(1):52-8

17. Anuradha C D, Kanno S, Hirano S. Fluoride induces apoptosis by caspase-3 activation in human leukemia HL-60 cells. Arch Toxicol 2000;74(4-5):226-30.

18. Singh A. Endemic fluorosis. Epidemiological, clinical and biochemical study of chronic fluorine intoxication in Punjab. Medicine 1961;42:229-246

19. Newman W F, Newman M W. The Chemical Dynamics of Bone Mineralization. University of Chicago Press, Chicago 1958

20. Parkin D M, Whelan S L, Ferlay J, Raymond L, Young J, eds. Cancer incidence in five continents. Volume VII. IARC Sci Publ 1997;(143):I-xxxiv,1-1240

21. Weinstock M A, Bogaars H A, Ashly M, Litle V, Bilodeau E, Kimmel S. Inaccuracies in certification of nonmelanoma skin cancer deaths. Am J Public Health 1992;82(2):278-81

22. Weatherbase.com

23. Fluoridation, Calgary Regional Health Authority, www.crha.org

24. Honolulu Board of Water Supply. www.hbws.org

25. Los Angeles Department of Water and Power. ladwp.com

26. Funari E, Bastone A, Bottoni P, De Donno D, Donati L. Quality problems in waters used for drinking purposes in Italy. Biomed Environ Sci 1991;4(4):339-47

27. Brothwell D J, Limeback H. Fluorosis risk in grade 2 students residing in a rural area with widely varying natural fluoride. Community Dent oral Epidemiol 1999;27(2):130-6

28. Attwood D, Blinkhom A S. Trends in dental health of ten-year old school children in south-west Scotland after cessation of water fluoridation. Lancet 1988;2(8605):266-7

29. Nell A, Sperr W. Analysis of the fluoride content of drinking water in Austria 1993. Wien Klin Wochenschr 1994;106(19):608-14

30. Pontie M, Rumeau M, Ndiaye M, Diop C M. The fluorosis problem in Senegal: data evaluation and presentation of a new method for defluoridating the water supply. Sante 1996;6(1):27-36

31. Somvanshi P R, Chaubey B S, Phadake R V, Sekharde P M. Fluorosis in Maharashtra. J Assoc Physicians India 1990;38(3):217-9

32. Poey J, Elsair J, Morgan P, Reggabi M, Hataab F. Evaluation of biologic balance as a function of radiologic status in a population living in a endemic fluoride zone of Southern Algeria. Eur J Toxicol Envim Hyg 1976;9(3):179-86

33. Susheela A K. Epidemiology and control of fluorosis in India. www.Nutrition Foundation.org/archives/apr84c.htm

34. Gopalakrishnan P, Vasan R S, Sarma P S, Nair K S, Thankappan K R. Prevalence of dental fluorosis and associated risk factors in Alappuzha district, Kerala. Natl Med J India 1999;12(3):99-103

35. Mann J, Mahmoud W, Ernst M, Sgan-Cohen H, Shoshan N, Gedalia I. Fluorosis and dental caries in 6-8-year-old children in a 5 ppm fluoride area. Community Dent Oral Epidemiol 1990;18(2):77-9

36. Lo G L, Bagramain R A. Prevalence of dental fluorosis in children in Singapore. Community Dent Oral Epidemiol 1996;24:25-7

37. al-Dashti A A, Willians S A, Curzon M E. Breast feeding, bottle-feeding and dental caries in Kuwait, a country with low-fluoride levels in the water supply. Community Dent Health 1995;12(1):42-7

38. Vigild M, Skougaard M, Hadi R A, al-Zaabi F, al-Yasseen I. Dental caries and dental fluorosis among 4-,6-,12-and 15-year-old children in kindergartens and public schools in Kuwait. Community Dent Health 1996;13(1):47

39. Banerjee S, ed. Fluorosis In India Institute of Social Sciences (New Delhi, India), Manuscript report 7 1992

40. Phantumvanit P. Fluoride mapping in Chiang Mai, Thailand. Osterreichische Zeitschrift fur Stomalogie 1982;79:303-4

41. Phantumvanit P, Songpaisan Y, Moller I J. A defluoridator for individual households. World Health Forum Vol. 9 1988

42. Shannon I L. Fluoride levels in drinking water in New Mexico. N M Dent J 1970;21(2):10-1

43. Rwenyonyi C, Bjorvatn K, Birkeland J, Haugejorden O. Altitude as a risk indicator of dental fluorosis in children residing in areas with 0.5 and 2.5 mg fluoride per liter in drinking water. Caries Res 1999;33(4):267-74

44. US Geological Survey, Washington D.C., 1967 Fluoride concentration in the United States.

45. NMED Drinking Water Bureau, New Mexico Department of Health, www.nmenv.state.nm.us/gwb/haflor.htm

46. Zohouri F V, Rugg-Gunn A J. Total fluoride intake and urinary excretion in 4-year-old Iranian children residing in low-fluoride areas. Br J Nutr 2000;83(1):15-25

47. Lakdawala D R, Puenkar B D. Fluorine content of water and commonly consumed foods in Bombay and a study of the dietary fluorine intake. Dent Dialogue 1974;1(3):16-22

48. Cao J, Zhao Y, Lin J. Brick tea consumption as the cause of dental fluorosis among children from Mangol, Kazak and Yugu populations in China Food Chem Toxicol 1997;35(8):827-33

49. Vainio H, Rautalahti M. An international evaluation of the cancer preventive potential of carotenoids. Cancer Epidemiol Biomarkers Prev 1998;7(8):725-8

50. Fujiki H, Suganuma M, Okabe S, Sueoka E, Suga K, Inai K, Nakachi K, Kimura S. Mechanistic findings of green tea as cancer preventive for humans. Proc Soc Exp Biol Med 1999;220(4):225-8

51. Steiner G G. The correlation between cancer incidence and kava consumption. Hawaii Med J 2000;59(11):420-2

52. Welch J T, Eswarakrishman S. Fluorine in bioorganic chemistry. 1991 John Wiley and Sons Inc. New York

53. O'dell B L, Sunde R A, eds. Handbook of Nutritionally Essential Mineral Elements. 1997 Marcell Dekker Inc. New York

I claim:

1. A method of preventing and treating cancer comprising the administration of a cancer chemopreventive and chemotherapeutic composition of matter to a mammal in need thereof in a sufficient amount to suppress the initiation, promotion, progression and result in the elimination of cancer with said composition comprising fluoride or a pharmaceutically suitable salt thereof.

2. The method of claim 1 wherein the composition is a solid.

3. The method of claim 1 wherein the composition is a liquid.

4. The method of claim 1 wherein the composition is administered orally.

5. The method of claim 1 wherein the composition is administered parentally.

6. The method of claim 1 wherein the composition is administered to a human.

7. The method of claim 1 wherein the composition includes the addition of calcium.

8. The method of claim 1 wherein the composition includes the addition of vitamin D.

9. The method of claim 1 wherein the composition includes the addition of calcium and vitamin D.
